(19) United States
(12) Patent Application Publication
(10) Pub. No.: US 2014/0356861 A1
(43) Pub. Date: Dec. 4, 2014

(54) METHOD FOR CHECKING THE SELF-HEALING PROCESS CARRIED OUT BY THE IMMUNE SYSTEM OF A TEST SUBJECT INFECTED WITH HUMAN PAPILLOMA VIRUS

(71) Applicant: Ralf Hilfrich, Huenfelden (DE)

(72) Inventor: Ralf Hilfrich, Huenfelden (DE)

(21) Appl. No.: 14/345,411

(22) PCT Filed: Sep. 19, 2012
(86) PCT No.: PCT/DE2012/100287

§ 371 Date: Mar. 18, 2014

(30) Foreign Application Priority Data
Sep. 19, 2011 (DE) 10 2011 053 741.4

(51) Int. Cl. G01N 33/569

(52) U.S. Cl. CPC G01N 33/56983; G01N 2800/7028
USPC 435/5

(57) ABSTRACT

A method and device for checking the self-healing process carried out by the immune system of a test subject infected with human papillomavirus (HPV) using one or more cell-containing samples taken from the test subject, to prevent the formation of an HPV-induced carcinoma. The method involves: (1) determining whether HPV with at least one HPV-specific stimulus molecule, stimulating the test subject's immune system, is contained in at least one of the samples; (2) determining whether innate HPV antibodies with the stimulus molecule are in the sample or at least one of the other samples; (3) analyzing results from (1) and (2) to determine whether the self-healing process has started based on the presence of the stimulus molecule and/or the antibodies.

### CROSS-REFERENCE TO RELATED APPLICATIONS

This application is a U.S. National Phase application under 35 U.S.C. §371 of International Application No. PCT/DE2012/100287 filed on Sep. 19, 2012, and claims benefit to German Patent Application No. DE 10 2011 053 741.4 filed on Sep. 19, 2011. The International application was published in German on Mar. 28, 2013, as WO 2013/041087 A1 under PCT Article 21(2).

### FIELD

The invention relates to a method for checking the self-healing process carried out by the immune system of a test subject infected with human papilloma virus

### BACKGROUND

Human papilloma viruses, abbreviated to HPV and also called HPV in the following, form a group of viruses which have meanwhile been classified into more than 100 types. The viruses attack the epithelial cells of the skin or various mucous membranes and cause an uncontrolled tumorous growth in the infected cells.

Some HPV types can cause malignant changes. For example, human papilloma viruses are suspected of being involved in the development of cancer of the neck of the uterus, so-called cervical cancer. Human papilloma viruses are also suspected of causing carcinomas of the vagina, penis and anus, or at least of being involved in the formation thereof.

Studies on subjects infected with HPV have shown that an infection with human papilloma viruses causing carcinoma by no means leads to a formation of carcinomas. The studies have shown that in many cases the immune system reacts to the HPV infection and initiates measures for destruction of the HPV. For example, the immune system activates so-called killer cells which destroy the human papilloma viruses early on, so that carcinoma formation is effectively counteracted. In this case a person infected with HPV will very highly probably not suffer from a carcinoma caused by HPV.

However, if the immune system does not react to such an HPV infection or at least does not react to the HPV infection with the appropriate measures, carcinoma formation highly probably occurs.

### SUMMARY

An aspect of the invention provides a method for checking the self-healing process carried out by the immune system of a test subject infected with human papilloma viruses (HPV), the self-healing process preventing HPV-induced carcinoma formation, the method comprising: providing a sample comprising cells taken from the test subject; (1) determining whether HPV comprising an HPV-specific stimulus molecule, which HPV-specific stimulus molecule stimulates the immune system of the test subject, is contained in the sample; (2) determining whether the sample comprises an endogenous antibody against the HPV; and (3) evaluating whether the HPV-specific stimulus molecule (i) and the endogenous antibody (ii) are present, such that, if (i) and (ii) are present, a beginning of the self-healing process carried out by the immune system of the test subject is confirmed.

### BRIEF DESCRIPTION OF THE DRAWINGS

The present invention will be described in even greater detail below based on the exemplary figures. The invention is not limited to the exemplary embodiments. All features described and/or illustrated herein can be used alone or combined in different combinations in embodiments of the invention. The features and advantages of various embodiments of the present invention will become apparent by reading the following detailed description with reference to the attached drawings which illustrate the following:

FIG. 1 a diagram describing a possible procedure in carrying out the method according to the invention for checking the self-healing process carried out by the immune system of a person infected with human papilloma viruses,

FIG. 2A to FIG. 2E method steps in carrying out a rapid test for the qualitative and/or quantitative determination of antibodies against human papilloma viruses contained in body fluid in the course of carrying out a method such as that based on the representation in FIG. 1.

### DETAILED DESCRIPTION

A further aspect of the invention provides a reliable prediction as to whether a carcinoma will also occur in a subject infected with human papilloma viruses.

A method according to an aspect of the invention for checking the self-healing process carried out by the immune system of a test subject infected with human papilloma viruses with the aid of one or at least two samples which are taken from the test subject and contain cells has the following steps:
1) determination of whether HPV having at least one HPV-specific stimulus molecule which stimulates the immune system of the test subject are contained in the sample or at least one of the samples;
2) determination of whether endogenous antibodies against the HPV having the stimulus molecule or against HPV having the stimulus molecule are contained in the sample or at least one other of the samples;
3) preferably evaluation of steps 1 and 2 to the effect that if the stimulus molecule is present and if the antibodies are present the self-healing process carried out by the immune system of the test subject is started, which prevents the formation of an HPV-induced carcinoma.

An aspect of the invention is based on the concept that checking of the self-healing process carried out by the immune system of a subject infected with HPV is based on two determining parameters, namely on the one hand whether the subject infected with HPV has an HPV-specific stimulus molecule which stimulates his immune system and on the other hand whether the subject infected with HPV has already formed endogenous antibodies which act against the HPV having the stimulus molecule.

It has been found that by methods according to the invention a method is realized by means of which it is predicted with high reliability whether in test subjects infected with HPV an HPV-induced formation of carcinomas occurs or whether such a carcinoma formation is prevented by the defense forces of the immune system.

A method according to the invention is suitable for use on all types of organisms. The method according to the invention can therefore in principle be employed both on humans and on animals.

A method according to the invention is suitable in particular for checking the self-healing process carried out by the immune system of a woman whose uterus, in particular whose cervix, is infected with HPV. By means of the method according to the invention, a reliable prediction can be made as to whether malignant tumours caused by HPV will form in the region of the uterus, in particular of the cervix. In this respect the method according to the invention is a particularly good aid in the decision as to whether a surgical intervention is to be performed on the woman, for example to perform a conisation.

A method according to the invention can be carried out as follows: With the aid of the sample taken, it is first investigated whether at least one of the cells removed with the sample is infected with HPV which has the stimulus molecule. If the test is positive, that is to say HPV having the stimulus molecule is found, it is investigated in the subsequent step whether the immune system of the test subject has already reacted to the HPV infection present by determining whether the immune system has already formed endogenous antibodies against the HPV having the stimulus molecule.

The same sample can be used to check whether endogenous antibodies have been formed. At least one further sample can also be taken from the test subject, for example at a later point in time. This checking can also be carried out, for example, with the aid of a sample which has been taken from a different place in the test subject to the preceding sample. In this respect, the sample can contain other body fluids. To carry out the checking of whether endogenous antibodies have been formed, in principle all types of body fluids can be used, such as, for example, blood, serum or plasma.

It is appropriate for the method according to the invention to be directed to at least one type of a capsid of the HPV as the stimulus molecule. Preferably, the L1 protein of the capsid should be used as the stimulus molecule. For example, the L1 protein of the HPV of type 16 can be used as the stimulus molecule. It is also conceivable to use the L2 protein of the capsid as the stimulus molecule.

Studies to date have shown that in HPV-infected patients whose cells still form the L1 protein formation of HPV-induced carcinomas is absent with a probability of more than 80%. When the immune system of the infected subject has formed endogenous antibodies against the L1 proteins and has reacted to the HPV infection in this manner, carcinomas have so far always remained absent.

The studies have furthermore shown that an HPV-induced formation of a carcinoma occurs in the HPV-infected subjects with a probability of more than 90% if the L1 stimulus is lacking and the immune system forms no endogenous antibodies against the L1 protein.

Against this background it is appropriate that it is first determined according to the invention whether HPV having at least one L1 protein as a stimulus molecule are contained in the sample and then, if HPV having the L1 protein are determined, it is checked whether the formation of endogenous antibodies against the L1 protein has started in the test subject.

The sample is preferably taken from the mucous membrane of the test subject. In this respect it is appropriate that for the sampling a smear is taken from a mucous membrane surface of the test subject, for example a smear from the mucous membrane of the cervix of a woman.

According to a further development of the invention, to determine whether HPV having the stimulus molecule are contained in the sample or at least one of the samples a step is carried out in which a detection of a morphological change and/or molecular biological change in at least one of the cells, preferably compared with a reference state, is carried out. Healthy test subjects are thereby detected, since their cells do not show a morphological change or molecular biological change. In this case further carrying out of the method according to the invention is not necessary, so that the further steps of the method according to the invention can be saved and a saving in costs thus results.

In order to distinguish that a change determined is a deviation compared with the healthy state, the morphological change and/or the molecular biological change is preferably ascertained by a comparison with healthy cells.

For detection of the morphological change and/or molecular biological change in the at least one cell, the sample can be subjected to an analysis which uses a change which is measurable and/or can be perceived by a user, preferably compared with a reference standard, in order to be able to draw therefrom conclusions regarding the morphological or molecular biological change in the cell compared with the healthy state of the cell, in particular in order thus to be able to determine such a change.

For this, an examination of the sample under a microscope can be performed, for example a morphological change in the at least one cell, preferably compared with a reference state, can be determined under the microscope after staining of the sample.

For detection of the morphological change in the at least one cell, it is appropriate for a Pap test to be carried out.

For detection of the molecular biological change in the at least one cell, it is possible, for example, for the sample to be subjected to an analysis in which a mutation of the cell and/or additional hereditary information, such as, for example, viral hereditary information, in the cell can be determined.

According to another further development of the invention, to determine whether the HPV having the stimulus molecule or HPV having the stimulus molecule are contained in the sample or at least one other of the samples a further step is carried out in which the morphological and/or molecular biological change in the at least one cell, preferably compared with a reference state, is identified by means of HPV-specific antibodies as an HPV-induced change in which HPV having the stimulus molecule are involved. A first estimation of whether formation of an HPV-induced carcinoma occurs in the HPV-infected subject can already be made by this further step.

Studies have shown that, for example, test subjects with an HPV infection in whom the stimulus molecule is not formed, that is to say the stimulus molecule is not determined, will highly probably suffer from a carcinoma. On the other hand, the test subject will highly probably form no HPV-induced carcinomas if the presence of the stimulus molecule in the sample is identified by means of this step. In this case, it is then determined in the subsequent step of the method according to the invention whether the immune system has already reacted to the stimulus molecule and endogenous antibodies against the HPV or the stimulus molecule are being formed.

The at least one HPV-specific antibody is preferably formed to bind to at least one HPV having the stimulus molecule, so that if the stimulus molecule is present, via the antibody the stimulus molecule bound with this can be concluded.

The HPV-specific antibody can be a non-human antibody. It is furthermore conceivable that the HPV-specific antibody is a human antibody. The HPV-specific antibody can also be an endogenous antibody.

It is appropriate that the HPV-specific antibody is a murine antibody, in particular a monoclonal murine antibody, since such an antibody can be prepared in a simple targeted manner for use in the method according to the invention.

In order to identify a capsid as the stimulus molecule, it is appropriate for the antibody to be a capsid-specific antibody. Preferably, the antibody is an L1-specific antibody, also called HPVL1-specific antibody in the following, in order to act as an antibody against the L1 protein of HPV.

The antibody can of course also be an L2-specific antibody, which then acts against the L2 protein of HPV.

According to one embodiment of the invention, it is provided that in the further step the sample is first heat-treated, the HPV-specific antibodies are then added and the sample with the HPV-specific antibodies is then subjected to an analysis which uses a change which is measurable and/or can be perceived by a user in the HPV-infected cells, preferably compared with a reference state, due to the addition of the HPV-specific antibodies.

Due to the heat treatment, existing protein structures are destroyed, as a result of which the binding of the antibodies to the cells infected with HPV is promoted. A so-called antigen demasking takes place due to the heat treatment. The treatment can be carried out by means of steam heat and/or microwaves. Preferably, the heat treatment of the sample is carried out at a temperature of between about 90 degrees and about 100 degrees Celsius, preferably at about 95 to 96 degrees Celsius.

It is appropriate for an amplifying agent to be added, by means of which the change which is measurable and/or can be perceived by a user in the cells infected with HPV due to the addition of the HPV-specific antibodies is amplified. The detection limit of the analysis by means of which the cells infected with HPV are identified due to the addition of the HPV-specific antibodies is lowered by the amplifying agent. In this respect the HPV having the stimulus molecule and the antibody adhering thereto can be identified particularly well by means of the amplifying agent.

It is furthermore appropriate for the amplifying agent to be bound to further HPV-specific antibodies and to be added together with the further HPV-specific antibodies, the further HPV-specific antibodies adhering to the HPV-infected cells and/or the HPV-specific antibodies already added. By this means a binding complex which comprises the cell infected with HPV, the HPV-specific antibody and the further HPV-specific antibody with the amplifying agent is produced in a simple manner.

The further HPV-specific antibody can be a non-human antibody. It is furthermore conceivable that the further HPV-specific antibody is a human antibody. The further HPV-specific antibody can also be an endogenous antibody.

It is appropriate that the further HPV-specific antibody is a murine antibody, in particular a monoclonal murine antibody, since such an antibody can be prepared in a simple targeted manner for use in the method according to the invention.

The further HPV-specific antibody is of course such an antibody which reacts to the stimulus molecule of the HPV and/or reacts to the HPV-specific antibody which is already adhering to the cell with the HPV and the at least one stimulus molecule.

The amplifying agent can be a molecule acting as a colour intensifier. The amplifying agent can be an enzyme, such as, for example, a peroxidase or phosphatase.

If the amplifying agent is a molecule acting as a colour intensifier, it is appropriate for the analysis to be carried out by addition of a predetermined amount of dyestuff, such as, for example, a chromogen, for staining of the at least one cell infected with HPV, in particular the at least one HPV-specific antibody adhering thereto. It is thereby possible to identify the HPV having the stimulus molecule in a technically simple manner, since the HPV-infected cell and/or the antibody adhering thereto are marked by staining.

In addition or alternatively, an analysis or identification of the HPV-infected cells or of the HPV-specific antibodies adhering thereto can be carried out by carrying out a radioactive labelling and/or identifying the HPV-infected cells or the HPV-specific antibodies adhering thereto by marking by means of fluorescent particles.

Preferably, the antibodies which are not adhering to the HPV-infected cells should be washed out. By this means it is ensured that the HPV-specific antibodies which are not adhering to the HPV-infected cells are washed out of the sample and do not enter into the analysis, so that, for example, in the course of a staining of the HPV-specific antibodies exclusively the antibodies adhering to the HPV-infected cells are rendered visible.

If in addition to the HPV-specific antibodies the further HPV-specific antibodies are also added, a washing out step should be carried out after each addition or a washing out should be carried out at the end at least of the least addition step. This is likewise against the background of having only the antibodies actually adhering to the HPV-infected cells in the sample, in order to identify in the course of the analysis, by means of marking thereof, in particular colour or fluorescence marking or labelling by means of radioactivity, the HPV-infected cells having the stimulus molecule.

According to a further development of the invention, it is provided that to determine whether endogenous antibodies against the HPV having the stimulus molecule are present, a test is carried out for the qualitative and/or quantitative determination of antibodies against HPV contained in body fluid, which comprises at least the following steps:
the sample is mixed with a reagent which substantially comprises a predetermined amount of physiologically acting liquid and a predetermined amount of at least one antigen which is specific with respect to the HPV having the stimulus molecule;
the mixture is then subjected to an analysis which uses a change which is measureable and/or can be perceived by a user, preferably compared with a reference state.

A detection of endogenous antibodies against HPV having the stimulus molecule can thereby be furnished in a simple manner and with little outlay.

For example, a rapid test for the qualitative and/or quantitative determination of antibodies against HPV contained in body fluid which is described in the German patent application with the application number 10 2010 061 028.3 of the same applicant as the present patent application can be used for this.

Preferably, this rapid test should be carried out with the analysis system described in the German patent application with the application number 10 2010 061 028.3, that is to say the analysis system described there should be used. In this respect, reference is made to the German patent application 10 2010 061 028.3 in its full scope and this in its full scope is a part of this patent application.

Due to the rapid test, an expensive and complicated analysis in a central laboratory is not necessary. In this respect the diagnostic investigation is already possible on site, that is to say in the practice of a registered doctor or in the hospital directly on the ward.

A saline solution, for example, can be used as the physiologically acting liquid in the course of the test.

Preferably, an HPV-specific antigen which identifies antibodies against HPV having the stimulus molecule is employed in the course of the test. If the stimulus molecule is, for example, an L1 protein, the HPV-specific antigen is an HPVL1-specific antigen, for example an HPV16L1-specific antigen which identifies antibodies against the L1 protein of the HPV of type 16.

If the HPV-specific antibodies against HPV having the stimulus molecule are determined in the sample by means of the test, these are those which have been formed by the immune system of the test subject, that is to say endogenous antibodies.

By the addition of the HPV-specific antigen, a complex of the antigen and any antibodies against the HPV having the stimulus molecule which are contained in the sample is already formed before the analysis. If the antibodies associated with the at least one antigen are present in the sample, the reagent is inactivated. If no corresponding antibodies are contained in the sample, the reagent remains reactive. This reactive property of the reagent is then utilized in the analysis.

Depending on whether or not antibodies against human papilloma viruses having the stimulus molecule are contained in the sample, a change which is measurable and/or can be perceived by a user occurs in the course of the analysis or such a change remains absent. In the test, by the analysis it is thus discovered by measurement or perception of a user whether the antibodies which are associated with the at least one antigen are present in the sample or are above the detection limit of the method or are not present or are below the detection limit of the method.

The change in the course of the analysis which can be perceived by the use can be a visual, acoustic and/or tactile change, preferably compared with a reference state. For example, a fluorescent medium can be used. The analysis can use a staining effect or discoloring effect or a color reaction. In principle, the analysis can use any type of measurable reaction. The analytical result can be rapidly determined by all of these possibilities.

Preferably, in the event of an absence of antibodies against the stimulus molecule or in the event of an amount of endogenous HPV antibodies in the sample which lies below or in the region of a predetermined detection limit for the HPV-specific antigen, a medium is changed in a manner which can be perceived by a user and/or measurably, preferably compared with a reference state, in particular over a predetermined section of the surface or a predetermined volume. The change can take place over a predetermined section of the surface of the medium or over a predetermined volume of the medium.

A predetermined amount of the mixture of the reagent and the sample of body fluid should be subjected to the analysis. Preferably, the predetermined amount should be a part amount of the mixture present. As a result, several diagnostic investigations can be carried out from the sample taken and the reagent combined with this, for example in order to acquire a statistical confirmation of the result.

The amount of the at least one HPV-specific antigen can furthermore be determined by a predetermined detection limit for the amount to be determined of the at least one HPV antibody present in the sample. The detection limit is preferably the particular desired detection limit, for example in order to ascertain that a particular amount of antibodies against HPV having the stimulus molecule is contained in the liquid sample, in order thus to confirm that the immune system has already responded to the presence of the HPV having the stimulus molecule and has reacted.

The invention also relates to a device for carrying out a method of the type described above.

The invention furthermore includes a use of an HPV-specific antibody for carrying out a method of the type described above, wherein the antibody binds to at least one HPV-infected cell of a test subject which, or the HPV of which, has at least one stimulus molecule which stimulates the immune system of an infected subject, in particular capsid, preferably L1 protein.

The invention furthermore includes a use of an HPV-specific antigen for carrying out a method of the type described above, wherein the antigen adheres to an endogenous antibody which is directed against HPV having a stimulus molecule which stimulates the immune system of an infected subject, in particular capsid, preferably L1 protein. The endogenous antibody in this context is the antibody formed by the immune system of the test subject infected with HPV in order to destroy the HPV having the stimulus molecule.

Further aims, advantages, features and possible uses of the present invention emerge from the following description of an embodiment example with the aid of the drawing. In this context, all the features described and/or shown in the figures by themselves or in any desired appropriate combination form the subject matter of the present invention, also independently of their combination in the claims or references back to them.

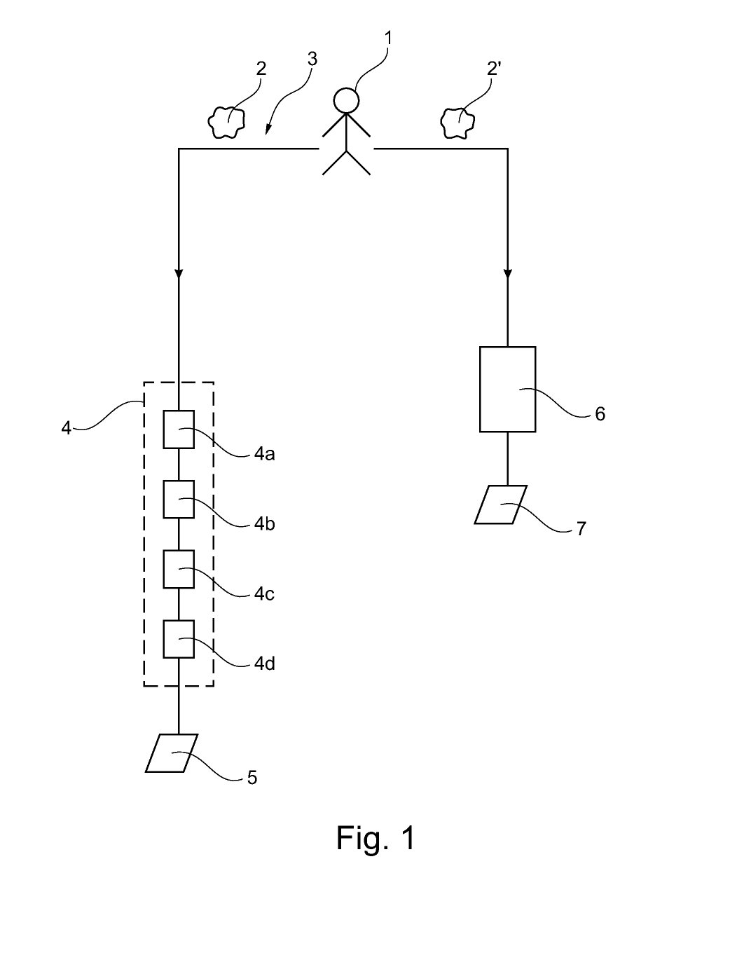

FIG. 1 shows a diagram of a possible procedure in carrying out the method according to the invention for checking the self-healing process carried out by the immune system of a person infected with human papilloma viruses (HPV).

According to the method, in a first step 3 a sample 2 with cells of a mucous membrane of a test subject 1 is taken from the test subject 1. For example, sampling can be by a smear of the surface of a mucous membrane of the test subject 1. The mucous membrane can be, for example, the mucous membrane of the uterus of a woman.

With the aid of the sample 2, it is determined in a further step 4 whether HPV having at least one HPV-specific stimulus molecule which stimulates the immune system of the test subject are contained in the sample 2. Preferably, for this it is determined whether the HPV have at least one L1 protein as the stimulus molecule.

If in the course of step 4 it is determined that as the result 5, HPV having the stimulus molecule is contained in the sample 2, this already fulfils a substantial prerequisite that the immune system of the test subject 1 acts against the HPV having the stimulus molecule and destroys the HPV and thus prevents the formation of an HPV-induced carcinoma.

It still remains to check whether the immune system of the test subject has in fact been stimulated by the stimulus molecule and was thus activated by the stimulus molecule in order to act against the HPV having the stimulus molecule.

For this a further sample 2′ is taken from the test subject 1, by means of which it is then determined whether the test subject has already formed endogenous antibodies against the HPV having the stimulus molecule.

This step 6 for checking for the presence of endogenous antibodies against the HPV having the stimulus molecule can be repeated several times, in particular by several time-delayed samplings 2′. However, it is also possible in principle for the checking of whether endogenous antibodies against the HPV having the stimulus molecule are already present also to be possible with the aid of sample 2, with the aid of which the preceding step 4 has already been carried out.

At the end of carrying out the method according to the invention, the conclusion can be drawn, as the result 7, that towards the HPV having the stimulus molecule which is present, the immune system of the test subject 1 has already formed endogenous antibodies against the HPV having the stimulus molecule. In this case the formation of an HPV-induced carcinoma is very highly probably prevented by the immune system of the test subject 1.

If the result 7 reads that the immune system of the test subject 1 has not responded to the HPV having the stimulus molecule, that is to say no endogenous antibodies against the HPV having the stimulus molecule are determined, the formation of an HPV-induced carcinoma will highly probably occur.

In the course of carrying out step 4, in which it is determined whether HPV having the stimulus molecule are contained in the sample 2, a first sub-step 4a is carried out, in which a detection of a morphological change and/or molecular biological change in at least one of the cells is carried out.

For this, the sample 2 with the cells is preferably applied to a microscope slide, in particular a glass microscope slide.

A so-called Pap test is then preferably carried out. For this, the cells contained in the sample 2 are stained by means of a dyestuff and the sample 2 with the stained cells is subjected to an evaluation under the microscope.

In the course of the evaluation under the microscope, morphological changes in the cells are searched for, for example, by a user.

If a such at least one morphological change in at least one cell of the sample is determined, it is to be checked whether this morphological change was caused by HPV, in particular HPV having the stimulus molecule.

Preferably, for this a further test is carried out in a further sub-step 4b of step 4. In the course of this test, the sample 2 is first subjected to an antigen demasking. For this, the sample 2 is heat-treated at approximately 95 degrees Celsius.

At least one, preferably several monoclonal murine antibodies of the same type are then added to the sample 2. The monoclonal murine antibody is preferably directed at adhering to the at least one HPV-infected cell having the stimulus molecule.

The heat treatment and the addition of the murine antibodies are in each case preferably carried out on the microscope slide with the sample 2.

Washing out of the murine antibodies is then carried out. For this, the murine antibodies which are still present in the free form, that is to say are not adhering to HPV-infected cells, are washed out from the microscope slide.

In a further step 4c, the murine antibodies adhering to the HPV having the stimulus molecule are now rendered visible. Preferably, for this a further murine antibody which likewise adheres to the HPV with the stimulus molecule or at least adheres to the previous murine antibodies already adhering to the HPV having the stimulus molecule is brought to the sample 2.

The further murine antibodies are bound to at least one enzyme, such as, for example, peroxidase or phosphatase, which intensifies the tendency of the complex of murine antibody and HPV stimulus molecule to become stained.

In a further step, the non-adhering constituents of the murine antibodies added are now in turn washed out from the sample 2 or from the microscope slide with the sample 2, so that exclusively murine antibodies which adhere to the cells with HPV and the stimulus molecule remain on the microscope slide with the sample 2.

A dyestuff, for example a chromogen, is now subsequently added to the sample 2, in particular the microscope slide with the sample 2, and, for example, subjected to an examination under the microscope.

Due to the addition of the dyestuff, under the microscope a precipitate staining is detectable where the stimulus molecule has formed a binding complex with the murine antibody and/or the further murine antibody with the enzyme bound therewith. Thus if such discolorations are to be detected under the microscope, it is thereby determined that HPV having the stimulus molecule are present in the sample 2.

The step 6 for determination of whether endogenous antibodies against the HPV having the stimulus molecule are formed by the test subject is preferably carried out by means of a rapid test for the qualitative and/or quantitative determination of antibodies against HPV contained in body fluid, such as is described in the German patent application 10 2010 061 028.3. Furthermore, an analysis system and a device for carrying out such a rapid test such as is likewise described in the German patent application 10 2010 061 028.3 is preferably employed for this.

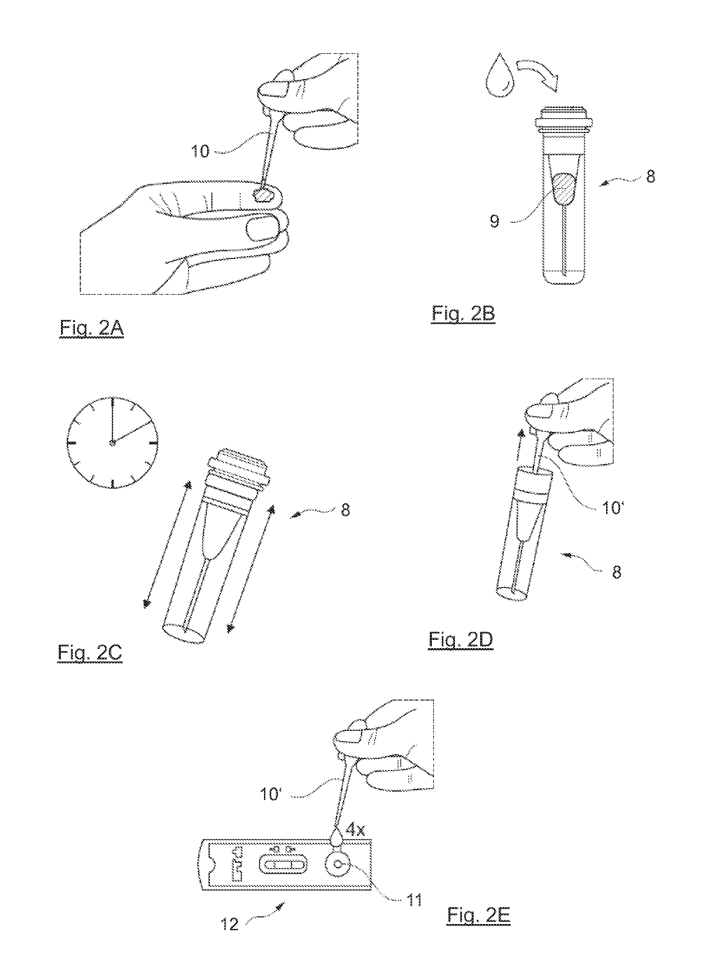

As can be seen from the example of FIGS. 2A to 2E, the sample 2′ can be taken by means of a pipette 10 (FIG. 2A). This can be a sample of whole blood or in turn a sample of another body fluid, such as, for example, mucus of a mucous membrane surface of the test subject.

The sample 2′ is introduced into a container 8, which contains a reagent 9, and is mixed with the reagent 9 (FIG. 2B). The reagent 9 substantially comprises a predetermined amount of physiologically acting liquid, such as, for example, saline solution, and a predetermined amount of at least one HPV-specific antigen.

In the present case, the HPV-specific antigen is such an antigen which identifies antibodies against HPV having the stimulus molecule and binds to these such antibodies.

If the stimulus molecule is an L1 protein, the antigen is an HPVL1-specific antigen.

For mixing the sample 2′ with the reagent 9, the container 8 is shaken several times and left to stand over a predetermined period of time, for example 10 minutes. Mixing of the amounts of sample with the reagent 9 has thereby taken place (FIG. 2C).

In a further step, the substrate now present in the container 8 is then at least partly removed by means of a pipette 10′ and a predetermined amount, for example one or more drops, of the substrate is fed to an analysis system 12 via a filling point 11. The analysis system 12 comprises a conjugate which substantially comprises a predetermined amount of a molecule which reacts to the stimulus molecule, the conjugate being marked with colloidal gold. Preferably, the molecule of the conjugate is an L1-specific molecule which in this respect identifies an L1 protein as the stimulus molecule.

After addition of the substrate to the analysis system 12, the following now happens (FIG. 2E):

If no amount of antibodies against HPV having the stimulus molecule above the detection limit of the analysis system 12 is present in the patient's sample, a discoloration in an envisaged region of the analysis system 12 occurs.

If antibodies against HPV having the stimulus molecule are present in the sample or are above the detection limit, such a discoloration is absent.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

### LIST OF REFERENCE SYMBOLS

1 Test subject
2 Sample
2′ Sample
3 Method step
4 Method step
4a Sub-step
4b Sub-step
4c Sub-step
4d Sub-step
5 Result
6 Method step
7 Result
8 Container
9 Reagent
10 Pipette
10′ Pipette
11 Filling point
12 Analysis system

1. A method for checking a self-healing process carried out by the immune system of a test subject infected with human papilloma viruses (HPV), the self-healing process preventing HPV-induced carcinoma formation, the method comprising:
providing a sample comprising cells taken from the test subject;
(1) determining whether HPV comprising an HPV-specific stimulus molecule, which HPV-specific stimulus molecule stimulates the immune system of the test subject, is contained in the sample;
(2) determining whether the sample comprises an endogenous antibody against the HPV; and
(3) evaluating whether the HPV-specific stimulus molecule (i) and the endogenous antibody (ii) are present, such that, if (i) and (ii) are present, a beginning of the self-healing process carried out by the immune system of the test subject is confirmed.

2. The method of claim 1, wherein the determining (1) comprises detecting morphological change, molecular biological change, or a morphological and molecular biological change in at least one of the cells.

3. The method of claim 2, wherein the determining (1) comprises conducting a Pap test and thereby detecting the morphological change.

4. The method of claim 2, wherein the determining (1) further comprises identifying a first HPC-specific antibody as the morphological, molecular biological, or morphological and molecular biological change.

5. The method of claim 4, further comprising:
(a) heat treating the sample;
(b) adding the first HPV-specific antibody, to obtain a mixture comprising the sample and the first HPV-specific antibody; and
(d) analyzing the mixture for a change in HPV-infected cells due to the adding (b) of the first HPV-specific antibody, the change being measurable, perceptible, or measurable and perceptible to a user.

6. The method of claim 5, further comprising:
(c) adding an amplifying agent to the mixture, thereby amplifying the change.

7. The method of claim 6, wherein the amplifying agent is bound to a second HPV-specific antibody,
wherein the amplifying agent is added together with the second HPV-specific antibody, and
wherein the second HPV-specific antibody adheres to the HPV-infected cells, the first HPV-specific antibody, or the HPV-infected cells and the first HPV-specific antibody.

8. The method of claim 6, wherein the amplifying agent is a molecule acting as a color intensifier, and
wherein the analyzing (b) comprises adding a predetermined amount of dyestuff suitable for staining
the HPV-infected cells,
HPV-specific antibody adhered to the HPV-infected cells, or
the HPV-infected cells and the HPV-specific antibody adhered to the HPV-infected cells.

9. The method of claim 5, further comprising:
removing an antibody not adhering to the HPV-infected cells.

10. The method of claim 1, wherein the determining (2) comprises testing a body fluid to provide a determination of HPV antibodies comprised in the body fluid, the testing comprising:
(i) mixing the body fluid with a reagent substantially comprising a physiologically acting liquid and an HPV-specific antigen, in predetermined amounts, to obtain a mixture;
(ii) analyzing the mixture for a change which is measurable, perceptible, or measurable and perceptible to a user.

11. The method of claim 10, characterized in that further comprising:
feeding a predetermined amount of the mixture to the analyzing (ii).

12. The method of claim 10, the amount of the HPV-specific antigen in the mixing (i) is determined by a predetermined detection limit for an HPV antibody amount in a fluid sample.

13. The method of claim 12, further comprising:
changing a medium of measurement such that a change can be measured, perceived, or measured and perceived by the user, if the analyzing (ii) determines the HPV antibody amount to be less than or approximately equal to the predetermined detection limit for the HPV-specific antigen.

14-16. (canceled)

17. The method of claim 9, wherein the removing comprises washing out the antibody not adhering to the HPV-infected cells.

18. The method of claim 10, wherein the determination is qualitative.

19. The method of claim 10, wherein the determination is quantitative.

20. The method of claim 13, wherein the medium is a predetermined surface section or a predetermined volume.

21. The method of claim 1, wherein the HPV-specific stimulus molecule comprises a capsid.

22. The method of claim 1, wherein the HPV-specific stimulus molecule comprises L1 protein.
